(19) United States
(12) Patent Application Publication
(10) Pub. No.: US 2015/0265209 A1
(43) Pub. Date: Sep. 24, 2015

(54) TECHNIQUES FOR MONITORING PRESCRIPTION COMPLIANCE USING A BODY-WORN DEVICE

(71) Applicant: Jack Ke Zhang, Ijamsville, MD (US)

(72) Inventor: Jack Ke Zhang, Ijamsville, MD (US)

(21) Appl. No.: 14/218,821

(22) Filed: Mar. 18, 2014

(51) Int. Cl. A61B 5/00; A61B 5/021; A61B 5/024

(52) U.S. Cl. CPC A61B 5/4833; A61B 5/024; A61B 5/021

(57) ABSTRACT

A method, apparatus, and/or system for monitoring therapy compliance is disclosed. In one step, therapy information specifying one or more medical treatments may be received. A regimen for the user may be retrieved, the regimen being based on the received therapy. An event may be generated, for example, on a body-worn device, depending on the regimen. At least one of the therapy, the regimen, or the event may be received by the body-worn device wirelessly. User input indicating compliance with the event may be received on the body-worn device. The user input may be recorded.

### BACKGROUND

This disclosure relates in general to medical therapy compliance and, but not by way of limitation, to systems and methods that are used to manage and monitor therapy compliance.

A patient undergoing medical treatment may often be prescribed one or more therapies and/or prescriptions by his or her physician. In the United States, it is estimated that 32 million people use three or more medications daily. Unfortunately, many people who are prescribed medication do not take it as directed by their doctor or pharmacist. In fact, as many as 75% of patients fail to adhere to, or comply with, physician-prescribed treatment regimens. Non-adherence examples include, but are not limited to, inadvertently taking the wrong dosage, forgetting to take the medication within a certain time period after eating, forgetting to take the medication entirely, forgetting to refill a prescription, and/or failing to take the medication for the recommended time period, to name a few. The estimated economic impact of non-adherence costs $100 billion annually.

Current techniques are lacking with regard to tracking the patient's compliance with these prescribed treatment regimens. For example, a patient may have to remember to take a pill at a particular time every day. Though various means may be used as reminders, the patient himself may typically be required to configure these reminders. Additionally, the patient may be required to manually document whether he, in fact, took the pill along with additional documentation. For example, a patient may have to manually measure his vital signs and document his results, presenting these results later to his physician. These procedures invite noncompliance given that a patient may make a myriad of mistakes including, but not limited to, inadvertently configuring the reminders incorrectly, failing to configure reminders, incorrectly measuring his vital signs, and/or forgetting to document his results. Additionally, significant periods of time elapse between medical appointments, making alterations to the regimen impractical unless the patient revisits his physician. With nation-wide healthcare, inefficiencies like this are borne by all through higher insurance premiums.

### SUMMARY

In one example embodiment, the present disclosure provides a computer-implemented method for managing medical therapy compliance using a body-worn device. The computer-implemented method includes receiving a therapy related to a user. The therapy specifies one or more treatments. A regimen for the user is received based on the therapy. An event is generated on the body-worn device, specified the regimen. At least one of the therapy, the regimen, or the event is received by the body-worn device wirelessly. User input is received indicating compliance with the event. The user input is recorded and reported wireless away from the body-worn device.

In another example embodiment, the present disclosure provides a body-worn device for managing therapy compliance. The body-worn device includes one or more processors and one or more memories coupled with the one or more processors. The one or more processors and one or more memories are configured to perform operations. The operations include receiving a therapy related to a user. The therapy specifies one or more treatments. A regimen for the user is retrieved based on the therapy. An event is generated on the body-worn device, specified by the regimen, the body-worn device receiving at least one therapy, the regimen, or the event wirelessly. User input is received indicating compliance with the event. The user input is recorded and reported wireless away from the body-worn device.

In yet another example embodiment, the present disclosure provides a non-transitory computer-readable storage medium for managing therapy compliance having computer-executable instructions stored thereon that, when executed by a processor, cause the processor to perform operations. The operations include receiving a therapy related to a user. The therapy specifies one or more treatments. A regimen for the user is retrieved based on the therapy. An event is generated on the body-worn device, specified by the regimen, the body-worn device receiving at least one therapy, the regimen, or the event wirelessly. User input is received indicating compliance with the event. The user input is recorded and reported wireless away from the body-worn device.

Further areas of applicability of the present disclosure will become apparent from the detailed description provided hereinafter. It should be understood that the detailed description and specific examples, while indicating various embodiments, are intended for purposes of illustration only and are not intended to necessarily limit the scope of the disclosure.

### BRIEF DESCRIPTION OF THE DRAWINGS

The present disclosure is described in conjunction with the appended figures:

FIG. 1 depicts an example environment of an embodiment of a therapy compliance engine;

FIG. 2 depicts an example system or architecture for providing a therapy compliance engine in accordance with at least one embodiment;

FIG. 3 depicts an example computer architecture for providing a therapy compliance engine, including a plurality of modules that may carry out various embodiments;

FIG. 4 illustrates a flowchart of an embodiment of a process for monitoring therapy compliance using a therapy compliance engine;

FIG. 5 depicts a block diagram of an example method for using the therapy compliance engine;

FIG. 6 depicts a block diagram of still one further example method for using the therapy compliance engine;

FIG. 7 depicts a block diagram of an example method for using the therapy compliance engine in the context of an emergency situation; and

FIG. 8 depicts an example user interaction in accordance with at least one embodiment.

It should be understood that the drawings are not necessarily to scale. In certain instances, details that are not necessary for an understanding of the invention or that render other details difficult to perceive may have been omitted. It should be understood that the invention is not necessarily limited to the particular embodiments illustrated herein.

### DETAILED DESCRIPTION

The ensuing description provides preferred exemplary embodiment(s) only, and is not intended to limit the scope, applicability, or configuration of the disclosure. Rather, the ensuing description of the preferred exemplary embodiment(s) will provide those skilled in the art with an enabling description for implementing a preferred exemplary embodiment. It should be understood that various changes could be made in the function and arrangement of elements without departing from the spirit and scope as set forth in the appended claims. Specific details are given in the following description to provide a thorough understanding of the embodiments. However, it will be understood by one of ordinary skill in the art that the embodiments may be practiced without these specific details.

As described in the background of this disclosure, embodiments of the present invention comprise methods for monitoring medical therapy compliance. Specifically, these methods include the use of a body-worn device, hereinafter the “device.” The device may be worn on any part of the body (e.g., a wrist). The device may include one or many sensors that may be used to track vital signs and/or locational information of the patient. As used herein, a “sensor” may comprise a heart-rate monitor, a blood pressure monitor, a glucose monitor, a global positioning system (GPS) device, a pedometer, or an altimeter. Additionally, the device may be capable of presenting notification to the user. These notifications may be audible, haptic, graphical, or textual in nature.

Generally speaking, embodiments of the present invention enable a patient to more effectively adhere to a physician-prescribed therapy using the body-worn device described above.

Embodiments for the present invention comprise body-worn devices and methods for managing medical therapy compliance. In at least one example, a body-worn device (e.g., a watch) is preconfigured with information regarding at least one therapy. For instance, the watch is preconfigured to be used for a blood pressure therapy. As used herein, a “therapy” may include one or more medical treatments including, but not limited to, one or more prescribed medications, one or more physical activities, one or more sensor documentation requests, or any combination of the above. In at least one example, information is loaded onto the device by a physician, a pharmacist, or another service provider. The pre-loaded information is then used to determine a regimen to be followed. A “regimen,” as used herein, is intended to mean a schedule specifying at least one situation for which at least one event associated with a therapy should be performed. For instance, a regimen may indicate that an event (e.g. medication intake) should occur at pre-determined periodic times. In at least one example, the regimen may indicate that an event (e.g., a prescription refill request transmission) ought to occur in response to a particular received input (e.g., indication that the patient has taken the last dosage in a prescription, by user request).

For example, consider the case in which a patient is diagnosed with high blood pressure. His physician prescribes medication A and instructs the patient to take 500 mg of medication A, twice daily, once in the morning, once in the evening, with each dose to be taken shortly after a meal. Additionally, the physician instructs the patient to manually take his blood pressure and document the results 3 times daily, equally spanned over the course of the day. In this example, the physician preconfigures a body-worn device (e.g., a watch) with this therapy. A regimen schedule is generated by the device based on the therapy. The regimen defines at least one day and time during which the patient should take his 500 mg of medication A. Additionally, the regimen specifies that the patient should be reminded to eat prior to taking his medication. For instance, a reminder is generated indicating that the patient should eat something. This reminder is presented to the user within some time prior to the time the patient is supposed to take medication A (e.g., 30 minutes). The regimen further specifies that blood pressure readings be taken some amount of time after ingestion of the medication (e.g., 1 hour). In at least one example, the device generates reminders including, but not limited to, a reminder to eat a meal, a reminder to take a medication, a reminder to take a sensor reading, or any suitable combination of the above.

In accordance with at least one embodiment, the device receives user input indicating compliance with the therapy. For instance, continuing with the previous example, the user is reminded to eat prior to taking his medication. Subsequent to the reminder being presented to the user, the user is prompted for input. The prompt may be included in the reminder or may exist as a separate prompt. In at least one example, the reminder constitutes a textual message presented on the device and/or an audible alert sounded by the device. The user acknowledges the reminder by dismissing the reminder and/or turning off the audible sound. In some cases, dismissing the reminder and/or turning off the audible sound may be considered user input indicating compliance with the reminder. In at least one example, the user is queried regarding his compliance. For instance, the user is posed the question “did you eat a meal?” The user enters input indicating either that he did eat a meal, or alternatively, that he did not eat a meal. In at least one example, a Bluetooth device is used to enter user input indicating compliance with the reminder. For instance, a medication container having Bluetooth communication capabilities sends, to the device, an indication that the medication container has been opened. This indication, alone or in combination with the reminder information, constitutes user input indicating that the user has complied with taking his medication.

In accordance with at least one embodiment, the device generates reminder events at the time the patient is supposed to take the medication. The user responds in a similar fashion as described above, by dismissing the reminder, turning off the audible sound, or affirmatively answering a question posed by the device. In at least one example, the regimen dictates that the device query the user with a question some period of time after the user has indicated that he has taken the medication. For instance, the user enters compliance input indicating that he has taken his blood pressure medication. The regimen specifies that one hour after receipt of the user compliance input the user be asked, “Are you feeling dizzy?” The user makes a selection on the device indicating a response to the question. The response is recorded by the device and reported, wirelessly, away from the device (e.g., to a server responsible for storing such information). Additionally, the regimen causes a blood pressure sensor to be activated some period after the user compliance input has been received. The period between user input and sensor activation may vary depending on the therapy. Alternatively, the device poses a question to the user to determine whether to initiate the sensor reading. For instance, the device poses the question “are you ready to take your blood pressure?” In at least one example, the user is required to indicate agreement before the sensor reading commences. The device records any sensor readings taken and reports the sensor readings away from the device (e.g., to a server responsible for storing such information).

In accordance with at least one embodiment, previously received user input is used to modify a regimen. User input, as described above, includes user actions taken in response to presented reminders, user actions taken regarding Bluetooth-enabled containers, user responses to questions posed by the device, and/or a lack of a user response. User input may be recorded by the device at any suitable time. In at least one example, the device reports user input electronically to a physician and/or pharmacist. This report may be reported in an email message, a text message, or any suitable type of electronic communication. Based on the report, or at any suitable time, the physician and/or pharmacist modify the prescribed therapy. This modification is electronically communicated to the device. In response to the modification, the device alters the therapy and/or regimen to reflect the modification. Additionally, or alternatively, the device modifies the regimen based on the received user responses in accordance with the therapy. For instance, the therapy is configured to adjust medication in response to a certain one or more user inputs. In one illustrative example, the regimen indicates that the user take 500 mg of medication A once a day. However, the user is posed a question such as “do you feel dizzy?” at some point in therapy, in accordance with the current regimen. The user indicates that he feels dizzy. Based on the affirmative response, the device automatically modifies the regimen such that the user is prompted to take another 500 mg dose of medication A. Alternatively, the device indicates to the user that he should refrain from taking any more medication. A variety of modifications may be determined and would depend on the particular therapy being implemented and the user input received.

In accordance with at least one embodiment, the device tracks the amount of medication that has been taken and/or that is remaining. For instance, a physician configures the device with a therapy that requires the patient to obtain 50 capsules of medication A. The therapy further specifies that the user should refill the prescription twice. Additionally, the physician configures the device with one or more pharmaceutical contacts. The device generates a prescription request and transmits such a request electronically using the prescription information as well as at least one of the pharmaceutical contacts. Alternatively, the device utilizes the GPS system to electronically transmit a prescription request to a pharmacy located within a particular radius of the patient's location (e.g., within 10 miles). Furthermore, the device prompts the user for input as to which pharmacy the patient wishes to send the prescription request. As the therapy progresses, user input is used to track the number of doses remaining before the patient runs out of medication. The device is configured to generate an electronic prescription request upon a determination that a particular number of doses remain. In one illustrative example, an electronic prescription request generates when the device detects or determines that the patient has 10 doses remaining.

In accordance with at least one embodiment, the device records information in the manner described above. Additionally, the device records user information including, but not limited to, medical allergy information, physician contact information, and emergency contact information. In at least one example, an emergency state is activated either by a sensor on the body-worn device or by the user. Upon entering the emergency state, the body-worn device reports information away from the device. In at least one example, the physician and/or emergency contact indicated in the recorded information is notified. Additionally, or alternatively, one or more emergency response units are notified of the user's emergency state. Another user, including, but not limited to, an emergency medical technician, or other health care provider, can access recorded information on the body-worn device. Additionally, or alternatively, the other user can select an option on the device that enables access of the recorded information on a device other than the body-worn device (e.g., a computer with a web browser).

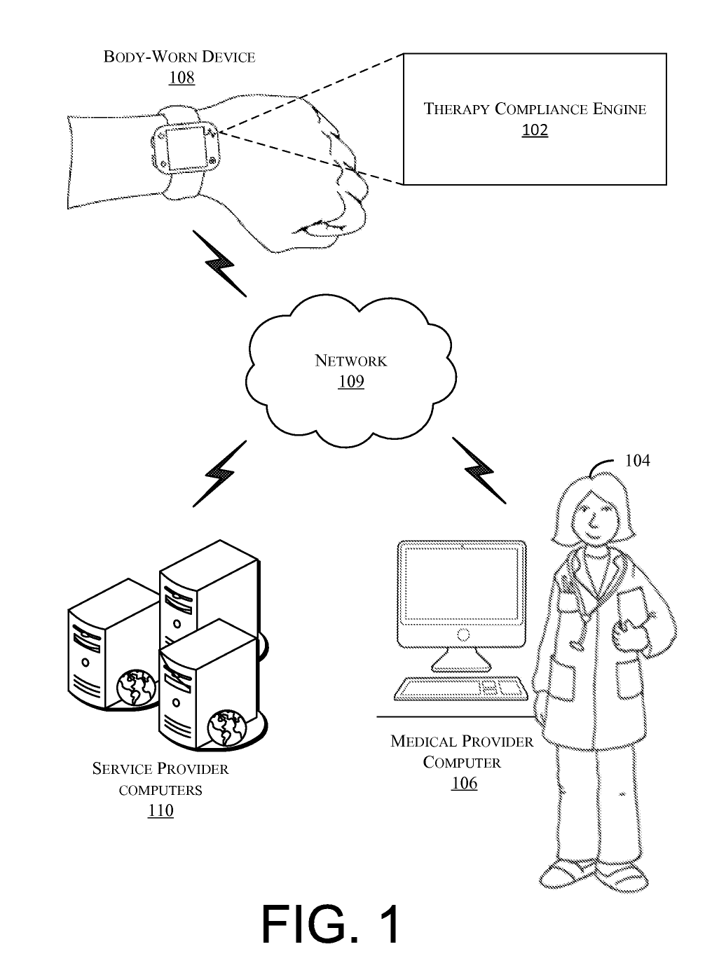

Referring now to the drawings, in which like reference numerals represent like parts, FIG. 1 depicts an example environment 100 of an embodiment of a therapy compliance engine 102. In accordance with at least one embodiment, a medical provider 104 (e.g., a physician or a pharmacist) configures a body-worn device 108 with a prescribed therapy. The medical provider 104 uses a medical provider computer 106 to input information associated with the therapy. Upon receipt, the information associated with the therapy is received by the therapy compliance engine 102.

In at least one embodiment, the therapy compliance engine 102 is a component of the body-worn device 108. Service provider computers 110 includes one or more computing devices responsible for storing and/or managing therapy information associated with one or more therapies. Service provider computers 110 communicate wirelessly with therapy compliance engine 102 to provide information regarding the therapy. This information includes therapy configuration. Additionally, as described above, the medical provider 104 utilizes medical provider computer 106 to modify a therapy. Such modifications are communicated to service provider computers 110. Service provider computers 110 records such modifications and communicates the modifications to therapy compliance engine 108. Therapy compliance engine 102 generates a new regimen or, alternatively, alters an existing regimen in accordance with the modifications.

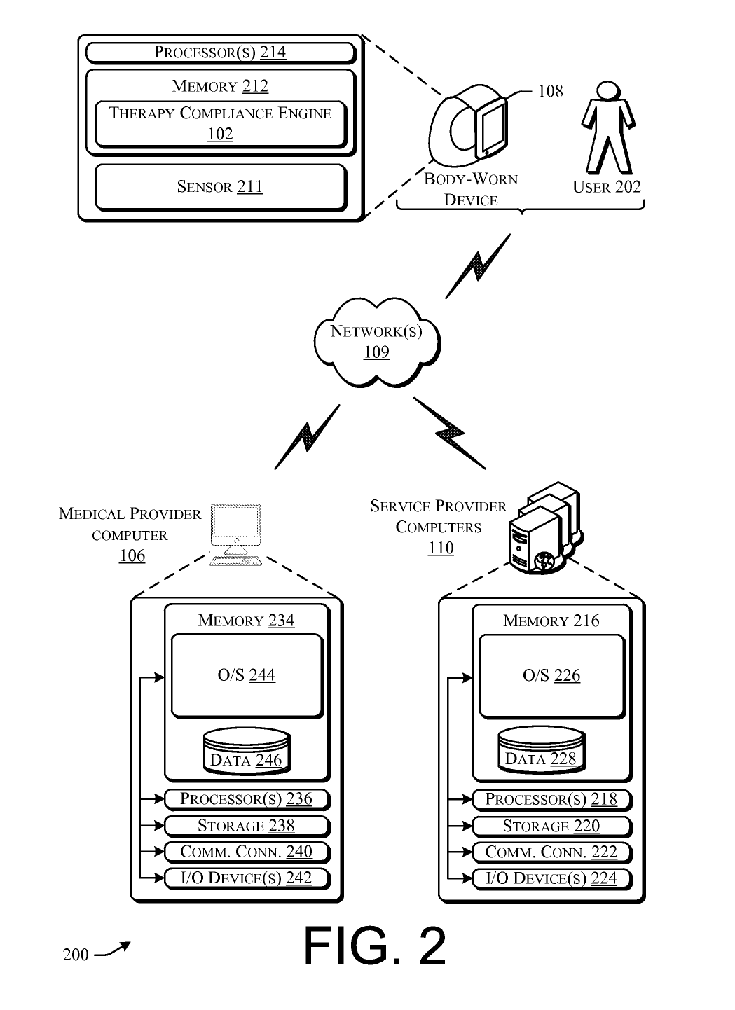

FIG. 2 depicts an example system or architecture 200 for providing the therapy compliance engine 102 in accordance with at least one embodiment. In architecture 200, a user 202 utilizes the body-worn device 108 (e.g., a body-worn watch) to access a therapy compliance engine 102, or a user interface accessible by the therapy compliance engine 102, via one or more networks 109. In some aspects, the therapy compliance engine 102 is hosted, managed, and/or provided by a computing resources service or service provider, such as by utilizing one or more service provider computers 110. The one or more service provider computers 110, in some examples, provide computing resources such as, but not limited to, client entities, low latency data storage, durable data storage, data access, management, virtualization, cloud-based software solutions, electronic content performance management, etc. The one or more service provider computers 110 are also operable to provide web hosting, computer application development, and/or implementation platforms, combinations of the foregoing, or the like to the user 202.

In some examples, the networks 109 include any one or a combination of many different types of networks, such as cable networks, the Internet, wireless networks, cellular networks and other private and/or public networks. While the illustrated example represents the user 202 accessing the therapy compliance engine 102 over the networks 109, the described techniques equally apply in instances where the user 202 interacts with the service provider computers 110 via the body-worn device 108 over a landline phone, via a kiosk, or in any other manner. It is also noted that the described techniques apply in other client/server arrangements (e.g., set-top boxes, etc.), as well as in non-client/server arrangements (e.g., locally stored applications, etc.).

As described briefly above, the therapy compliance engine 102 allows the user 202 to interact with the service provider computers 110. The one or more service provider computers 110, perhaps arranged in a cluster of servers or as a server farm, host the therapy compliance engine 102 and/or cloud-based software services. Other server architectures are used to host the therapy compliance engine 102 and/or cloud-based software services. The therapy compliance engine 102 is capable of handling requests from a user 202 and serving, in response, various user interfaces that are rendered at the body-worn device 108 such as, but not limited to, perceived latency or the like. The therapy compliance engine 102 provides any type of device or application control. The therapy compliance engine 102 and/or corresponding control are provided by the Operating System of the body-worn device 108. As discussed above, the described techniques can similarly be implemented outside of the therapy compliance engine 102, such as with other applications running on the body-worn device 108.

The body-worn device 108, in at least one example, is a computing device such as, but not limited to, a watch, a necklace, a mobile phone, a smart phone, a personal digital assistant (PDA), a thin-client device, a tablet PC, an electronic book (e-book) reader, or any suitable device capable being worn on a person and capable of communicating with a sensor 211. The sensor 211 includes at least one of a heart-rate monitor, a blood pressure monitor, a glucose monitor, a GPS device, a pedometer, or an altimeter. In some examples, the body-worn device 108 is in communication with the service provider computers 110 via the networks 109, or via other network connections. Additionally, the body-worn device 108 may be part of a distributed system managed by, controlled by, or otherwise part of the service provider computers 110.

In one illustrative configuration, the body-worn device 108 includes at least one memory 212 and one or more processing units (or processor(s)) 214. The processor(s) 214 is implemented as appropriate in hardware, computer-executable instructions, firmware, or combinations thereof. Computer-executable instruction or firmware implementations of the processor(s) 214 include computer-executable or machine-executable instructions written in any suitable programming language to perform the various functions described.

The memory 212 stores program instructions that are loadable and executable on the processor(s) 214, as well as data generated during the execution of these programs. Depending on the configuration and type of body-worn device 108, the memory 212 may be volatile (such as random access memory (RAM)) and/or non-volatile (such as read-only memory (ROM), flash memory, etc.). The body-worn device 108 also includes additional removable storage and/or non-removable storage. The removable and/or non-removable storage may provide non-volatile storage of computer-readable instructions, data structures, program modules, and other data for the computing devices. In some implementations, the memory 212 includes multiple different types of memory, such as static random access memory (SRAM), dynamic random access memory (DRAM), or ROM.

Turning to the contents of the memory 212 in more detail, the memory 212, in at least one embodiment, includes an operating system and one or more application programs, modules, or services for implementing the features disclosed herein including at least the perceived latency, such as via the therapy compliance engine 102 or dedicated applications. In at least one example embodiment, the therapy compliance engine 102 is configured to receive, store, and/or display content and at least interface for interacting with the service provider computers 110 and/or user 202. Additionally, the memory 212 stores access credentials and/or other user information such as, but not limited to, user IDs, passwords, and/or other user information. In some examples, the user information includes information for authenticating an account access request such as, but not limited to, a device ID, a cookie, an IP address, a location, or the like. Additionally, the user information includes information regarding a therapy associated with user 202.

In some aspects, the service provider computers 110 and medical provider computer 106 are any type of computing devices such as, but not limited to, a mobile phone, a smart phone, a personal digital assistant (PDA), a laptop computer, a desktop computer, a server computer, a thin-client device, a tablet PC, etc. Additionally, it should be noted that in some embodiments, the service provider computers 110 and/or medical provider computer 106 are executed by one or more virtual machines implemented in a hosted computing environment. The hosted computing environment may include one or more rapidly provisioned and released computing resources, which computing resources may include computing, networking and/or storage devices. A hosted computing environment is also referred to as a cloud-computing environment. In some examples, the service provider computers 110 and/or medical provider computer 106 are in communication with the body-worn device 108 and/or other service providers via the networks 109, or via other network connections. In at least one example, the service provider computers 110 includes one or more servers, perhaps arranged in a cluster, as a server farm, or as individual servers not associated with one another. These servers are configured to implement the therapy compliance engine 102 described herein as part of an integrated, distributed computing environment.

In one illustrative configuration, the service provider computers 110 and medical provider computer 106 each include at least one memory (e.g., the memory 216 and the memory 234) and one or more processing units (e.g., processor(s) 218 and processor(s) 236). The processor(s) 218 and/or the processor(s) 236 are implemented as appropriate in hardware, computer-executable instructions, firmware, or combinations thereof. Computer-executable instruction or firmware implementations of the processor(s) 218 and the processor(s) 236 include computer-executable or machine-executable instructions written in any suitable programming language to perform the various functions described.

In at least one example embodiment, the memory 216 and/or the memory 234 store program instructions that are loadable and executable on the processor(s) 218 or the processor(s) 236, respectively, as well as data generated during the execution of these programs. Depending on the configuration and type of service provider computers 110 and/or medical provider computer 106, the memory 216 and/or the memory 234 may be volatile (such as RAM) and/or non-volatile (such as ROM, flash memory, etc.). The service provider computers 110 and/or the medical provider computer 106 also include additional storage (e.g., additional storage 220 and additional storage 238) which includes removable storage and/or non-removable storage. The additional storage 220 and the additional storage 235 include, but are not limited to, magnetic storage, optical disks and/or tape storage. The disk drives and their associated computer-readable media provide non-volatile storage of computer-readable instructions, data structures, program modules and other data for the computing devices. In some implementations, the memory 216 and the memory 234 include multiple different types of memory, such as SRAM, DRAM, or ROM.

The memory 216, the memory 234, the additional storage 220, the additional storage 238, both removable and non-removable, are all examples of computer-readable storage media. In at least one example, computer-readable storage media include volatile or non-volatile, removable or non-removable media implemented in any method or technology for storage of information such as computer-readable instructions, data structures, program modules, or other data. The memory 216, the memory 238, the additional storage 220, and the additional storage 238 are all examples of computer storage media. Additional types of computer storage media that may be present in the service provider computers 110 and/or medical provider computer 106 may include, but are not limited to, PRAM, SRAM, DRAM, RAM, ROM, EEPROM, flash memory or other memory technology, CD-ROM, DVD or other optical storage, magnetic cassettes, magnetic tape, magnetic disk storage or other magnetic storage devices, or any other medium which can be used to store the desired information and which can be accessed by the service provider computers 110 and/or medical provider computer 106, respectively. Combinations of any of the above should also be included within the scope of computer-readable media.

In accordance with at least one embodiment, the service provider computers 110 and/or medical provider computer 106 contain communications connection(s) (e.g., 222 and 240) that allow the service provider computers 110 and/or medical provider computer 106 to communicate with a stored database, another computing device or server, user terminals and/or other devices on the networks 109. The service provider computers 110 and/or medical provider computer 106 also include I/O device(s) 224 and/or I/O device(s) 242, respectively, such as a keyboard, a mouse, a pen, a voice input device, a touch input device, a display, speakers, a printer, etc.

Turning to the contents of the memory (e.g., the memory 216 and/or the memory 234) in more detail, each memory includes an operating system (e.g., 226 and 244), one or more data stores (e.g., 228 and 246), and/or one or more application programs, modules, or services for implementing the features disclosed herein.

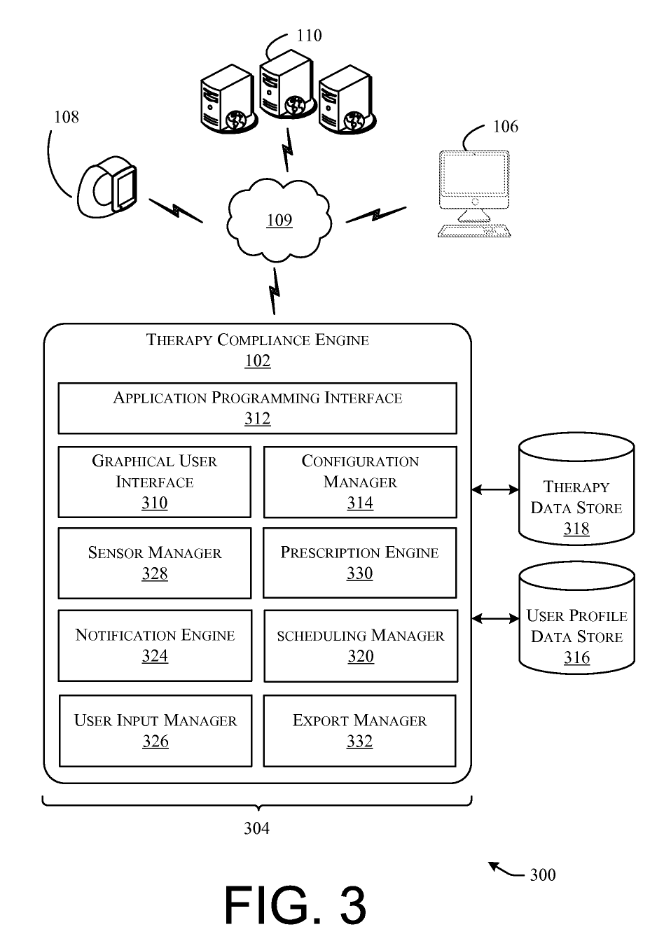

FIG. 3 depicts an example computer architecture 300 for providing a therapy compliance engine 102 including a plurality of modules 304 that may carry out various embodiments. In at least some examples, the modules 304 are software modules, hardware modules, or a combination thereof. If the modules 304 are software modules, the modules 304 are embodied on a computer-readable medium and processed by a processor in any of the computer systems described herein. It should be appreciated that any module or data store described herein, may be, in some embodiments, a service responsible for managing data of the type required to make corresponding calculations. The modules 304 may be configured in the manner suggested in FIG. 3 or may exist as separate modules or services external to the therapy compliance engine 102.

In accordance with at least one embodiment, a method is enabled for managing therapy compliance using a body-worn device. For example, the therapy compliance engine 102 is a component of the body-worn device 108 as discussed above in connection with FIG. 2. The therapy compliance engine 102 is stored on body-worn device 108 or, alternatively, is stored on a server accessible to the body-worn device 108 via network 108. An administrator (e.g., a physician) configures the therapy compliance engine 102 via a graphical user interface 310, a component of the therapy compliance engine 102. The administrator enters configuration information via medical provider computer 106, the configuration information including, but not limited to, data pertaining to one or more therapies, information associated with a user of the body-worn device 108 (e.g., a patient), and information regarding one or more pharmaceutical contacts. Once configuration information is entered via graphical user interface 310, application programming interface 312, a component of the therapy compliance engine 102, is utilized to communicate the configuration information to the therapy compliance engine 102.

In accordance with at least one embodiment, configuration manager 314, a component of the therapy compliance engine 102, is configured to receive configuration information. The configuration manager 314 is responsible for creating and maintaining a user profile utilized to store such configuration information. Further, the configuration manager 314 causes such configuration data to be stored in a user profile data store 316. Additionally, or alternatively, configuration manager 314 interacts with therapy data store 318, a data store responsible for storing information regarding one or more therapies. In at least one example, the configuration manager 314 queries therapy data store 318 for information regarding one or more therapies indicated in the received configuration information. Any information returned from therapy data store 318 is stored by the configuration manager 314 in user profile data store 316, along with, or separate from, the user profile.

In at least one embodiment, scheduling manager 320 is configured to receive configuration information including information pertaining to a prescribed therapy. The scheduling manager 320 is responsible for generating a regimen based on the prescribed therapy. The regimen indicates one or more notifications to be provided to the user at a specific day and/or time. The regimen additionally indicates one or more particular times at which to transmit reports to service provider computers 110. In at least one example, scheduling manager 320, according to the generated regimen, causes notification engine 324 to provide one or more electronic notifications on body-worn device 108. The notification may include, but is not limited to, a reminder to eat a meal, to take a dosage of medication, or to conduct a form of exercise.

In at least one embodiment, user input manager 326 is configured to present questions to the user via body-worn device 108. In at least one example, scheduling manager 320 determines one or more questions to be posed to the user at a particular time in accordance with the generated regimen. In such a case, scheduling manager 320 causes user input manager 326 to pose the determined question(s) to the user via body-worn device 108 at the appropriate time. The user utilizes body-worn device 108 to respond to the question(s). Upon receipt of the response, user input manager 326 stores such response data in user profile data store 326. Additionally, user input manager 326 causes scheduling manager 320 to act upon the response in one or more ways based on the therapy implemented. In one example, scheduling manager 320, determining that it is time for the user to take a dose of medication, causes notification engine 324 to present a reminder to the user on body-worn device 108. The user input manager 326 sends to the device a question such as “did you take your medication?” The user responds affirmatively or negatively. Alternatively, the user, having had no question posed, affirmatively indicates, via body-worn device 108, that he took his medication at a particular time. Either or both user inputs are received by user input manager 326. Additionally, such user input is stored in user profile data store 316 and is forwarded to scheduling manager 320. Scheduling manager 320, in response to such user input, updates the regimen.

In at least one example, scheduling manager 320 causes user input manager 326 to pose a question to the user via body-worn device 108. For instance, scheduling manager 320 determines that a question ought to be posed to the user at a particular time, or because of a particular response. For instance, the regimen may specify that the user be asked, “Are you feeling light-headed?” an hour after the user has indicated that he took his medication. In such a case, scheduling manager 320 causes user input manager 326 to pose the question to the user via device 326. The user responds to the question via body-worn device 108 and such response is received by user input manager 326, stored in user profile data store 316, and/or forwarded to scheduling manager 320. In at least one embodiment, scheduling manager 320 updates the regimen based on the response. For example, the therapy may indicate that, if the user responds that he does, in fact, feel light-headed when asked (e.g., an hour after taking his medication), the regimen be altered in some way (e.g., by increasing or decreasing the medication dosage). In at least one example, the regimen is altered such that the user is immediately prompted to take an additional dosage. Furthermore, the regimen is updated by the scheduling manager 320 to reflect changes brought on by the received user input.

In at least one embodiment, a therapy may specify one or more times for which a sensor contained in body-worn device 108 may be used to ascertain one or more patients' vital signs. For instance, a therapy specifies that the user's pulse and blood pressure should be taken once every hour. Such specifications are included in the regimen generated by scheduling manager 320. The therapy additionally, or alternatively, indicates certain chains of events that should result in activation of the sensor(s). For instance, a user is reminded to take his medication. He, in fact, takes the medication and responds to the reminder, or a posed question, indicating that he took his medication. Upon this input, or some time later, scheduling manager 320 causes sensor manager 328, a component of therapy compliance engine 102, to activate one or more sensors located on body-worn device 108. Sensor manager 328 communicates with the one or more sensors to cause vital sign information to be collected. For instance, in the ongoing example, sensor manager 328 causes a heart rate sensor to be activated. The sensor manager 328 is configured to receive data from the heart rate sensor. The sensor manager 328 further causes the heart rate information to be stored in user profile data store 316 and/or forwards the heart rate information to the scheduling manager 320 for analysis. Sensor manager 328, additionally or alternatively, activates blood pressure sensor. The sensor manager 328 is configured to receive data from the blood pressure sensor. The sensor manager 328 causes the blood pressure sensor to be stored in user profiled data store 326 and/or forwards the blood pressure information to the scheduling manager 320. Scheduling manager 320, as discussed above, analyzes the heart rate information and/or the blood pressure information to determine any regimen modification(s) necessary in accordance with the therapy. Though a heart rate sensor and a blood pressure sensor are used in this example, it should be appreciated that any sensor, or combination of sensors, able to communicate with body-worn device 108 may be utilized, in any suitable order, via a similar manner as described above.

In at least one embodiment, sensor manager 328 is configured to receive data from a sensor that is passively monitoring vital signs of the user. For instance, a heart rate sensor can passively monitor the wearer's hear rate. The sensor manager 328 is configured to receive such information and analyze the sensor data. The sensor manager 328 is configured to determine when the heart rate has indicated an adverse condition. For example, perhaps the user's heart rate drops dangerously low, or even stops. The sensor manager 328 receives such information and determines that the rate is in an unacceptable range. Upon such a determination, the sensor manager 328 causes notification engine 324, or any other suitable component of the therapy compliance engine 102, to access the user profile data store 316 for user profile data. User profile data indicates physician contact information and/or emergency contact information, for example. If the user profile data includes such information, the notification engine 324 may cause a notification to be sent to the indicated physician/emergency contact. In at least one example, the notification includes an automated phone call, email message, text message, or other suitable form of communication. Additionally, or alternatively, the notification engine 324 reports the adverse condition to an emergency response unit. In one example, upon determining the existence of an adverse condition, the sensor manager 328 causes the GPS to activate to ascertain the user's location. Any other sensor, or combination of sensors, included on the device may be similarly activated. Information collected by the sensor(s) is received by the sensor manager 328. The sensor manager 328 can relay the information to notification engine 324. Notification engine 324 may then report such information away from the device in a manner similar to that described above.

In at least one embodiment, the user may activate a setting on the device to indicate an emergency status. For example, the user may be aware that they are having a health issue and interact with a user interface (e.g., a button) located on the device. The indication is received by the user input manager 326. User input manager is configured to access user profile data store 316 to obtain user profile data in order to determine contact information similar to that described in the previous example. User input manager 326 is configured to cause notification engine 324 to notify the determined contacts and/or emergency response unit(s).

In at least one embodiment, another user may access information contained on and/or recorded by the device. For example, in an emergency situation, another user can access medical allergy information of the user. Additionally, or alternatively, someone other than the user may access information recorded by the device. As an example, a physician can choose a setting on the device that will enable therapy compliance information to be displayed on the device. The activation of such a setting is received by the user input manager 326. The user input manager 326 accesses user profile data store 316 to obtain therapy compliance information for the user. The user input manager 324 can then display such information on the device and/or enable the physician to access such information at a remote location (e.g., a website).

In at least one embodiment, a scheduling manager 320 determines, at the beginning of a therapy, or at any suitable time during the therapy, that a prescription request ought to be transmitted. A prescription request contains information about the patient and the treatment necessary for a pharmacy and/or health care provider to fulfill a prescription and/or schedule an appointment. For example, the therapy indicates that a patient has three refills on a given prescription and that the patient should take this medication until he has exhausted all three prescriptions. In accordance with the generated regimen, scheduling manager 320 causes prescription engine 330 to generate a prescription request. In at least one example, the prescription engine 330 ascertains medication information from the scheduling manager 320, therapy data store 328, and/or user profile data store 316. In at least one example, patient information, including contact information of a preferred pharmacist is ascertained from user profile data store 326 and utilized to generate an electronic prescription request to be transmitted to the preferred pharmacist regarding the medication information. The prescription engine 330 utilizes application programming interface 312 and/or a form of electronic communication (e.g., email, text messaging) to transmit the prescription request. Prescription engine 330 is configured to receive a response indicating that the prescription request has been fulfilled. For example, the recipient of the prescription request responds with an email, text message, or some other suitable form of electronic communication, indicating that the prescription has been fulfilled and is ready for pickup. The prescription engine 330, upon receipt of such information, cause notification engine 324 to provide the user with a reminder to pick up the medication.

In at least one embodiment, prescription engine 330 is used to request a prescribed treatment. For instance, the therapy indicates that the user should participate in physical therapy with a medical provider. In accordance with the generated regimen, scheduling manager 320 causes prescription engine 330 to send a request to schedule one or more therapy sessions.

In accordance with at least one embodiment, scheduling manager 320 determines, based on the current regimen, that patient compliance information (e.g., user input, user responses, vital sign information) should be sent to a medical provider (e.g., the prescribing physician). Additionally, or alternatively, scheduling manager 320 receives input requesting a compliance report including the patient compliance information. In either case, scheduling manager 320 causes export manager 332 to compile a report including patient compliance information and electronically transmit it to a particular location. In at least one example, the report is emailed to the prescribing physician.

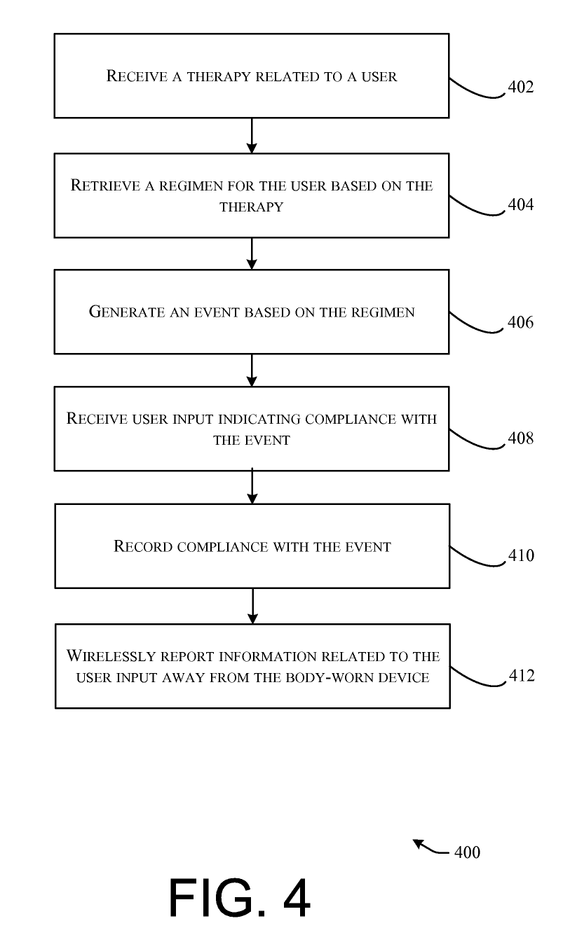

FIG. 4 illustrates a flowchart 400 of an embodiment of a process for managing therapy compliance using a therapy compliance engine (e.g., the therapy compliance engine 102). The flow chart 400 begins at 402, where a therapy related to a user is received. As described above, a “therapy” includes, but is not limited to, one or more prescribed medications, one or more physical activities, one or more sensor requests, or any combination of the above. A user is a patient recipient of the therapy. The therapy is received by a body-worn device. The body-worn device (e.g., the body-worn device 108 of FIG. 2) may include one or many sensors (e.g., sensor 211) that are used to track vital signs of the patient and/or locational information. As described above, a “sensor” may comprise a heart-rate monitor, a blood pressure monitor, a glucose monitor, a global positioning system (GPS) device, a pedometer, or an altimeter. Additionally, the body-worn device may be capable of presenting notification to the user (e.g., via notification engine 324). These notifications may be audible, haptic, graphical, or textual in nature.

At 404, a regimen for the user is retrieved based on the therapy. For instance, the regimen is determined by the scheduling manager 320 of FIG. 3 in the manner described above. Alternatively, the regimen is retrieved from therapy data store 318 of FIG. 3. As described above, the regimen specifies at least one situation for which at least one event associated with a therapy should be performed. For instance, a regimen indicates that an event (e.g. medication intake) should occur at pre-determined periodic times. In at least one example, the regimen indicates that an event (e.g., a prescription refill request transmission) ought to occur in response to a particular received input (e.g., indication that the patient has taken the last dosage in a prescription). A variety of situations exist and would depend on the therapy being implemented.

At 406, an event is generated based on the regimen. The event includes a reminder (e.g., a reminder to take medication), a request (e.g., a prescription request), and/or a question to be posed to the user (e.g., “Do you feel out of breath?”). The event is presented on the body-worn device either audibly, and/or textually.

At 408, user input is received, the user input indicating compliance with the event. The indication of compliance may be positive or negative. The user input includes input entered by the user via the body-worn device. Additionally, the user input includes information received from a Bluetooth-enabled device (e.g., a Bluetooth-enabled medication bottle).

At 410, compliance with the event is recorded. In at least one example, event compliance is recorded in user profile data store 316 of FIG. 3. In another example, event compliance is recorded and transmitted via a network to a medical provider computer 106.

At 412, information related to the user is wirelessly reported away from the body-worn device 108. In at least one example, this information relates to the user's compliance with a therapy. The information is reported to service provider computers 110 and/or medical provider computer 106. Alternatively, the information is sent wirelessly, to a physician via email.

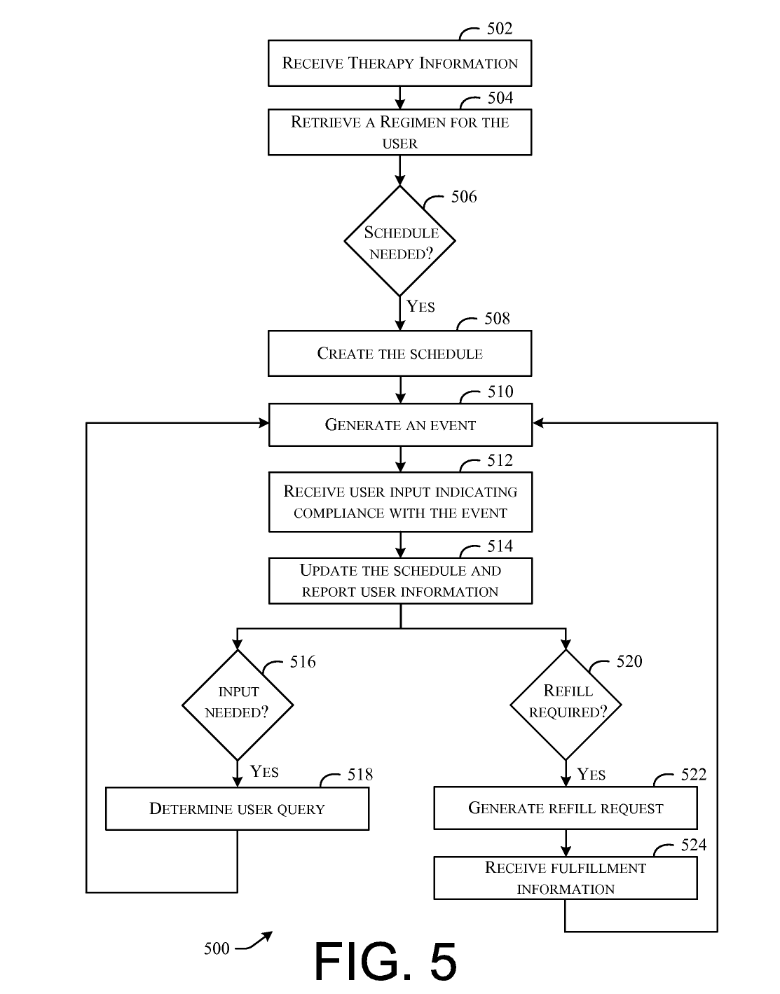

FIG. 5 depicts a block diagram 500 of another example method for using the therapy compliance engine 102. The method begins at block 502 where therapy information is received by a therapy compliance engine (e.g., the therapy compliance engine 102). At block 504, a regimen is retrieved for the user. In at least one example, the retrieved regimen is based on the received therapy. At decision point 506, the therapy compliance engine makes a determination as to whether a schedule is necessary. This determination is based on information regarding the received therapy. If a schedule is needed, the method continues to block 508 where a schedule is created that specifies at least one situation for which at least one event associated with a therapy should be performed. At block 510, an event is generated. In at least one example, the event is determined by the regimen. At block 512, user input indicating compliance with the event is received. As discussed above, compliance is received from the user via a body-worn device or, alternatively, from a Bluetooth-enabled device. At block 514, the schedule is updated and information related to the user (e.g., information indicating user compliance) is reported out. As described above, updating the schedule occurs because of received user input. At decision point 516, the therapy compliance engine determines whether user input is needed. This determination is based on the therapy and/or the schedule. If user input is determined to be necessary at block 516, one or more user queries are determined at block 518. The determination of the one or more user queries depends on the therapy and/or the schedule. If a user query is determined, an event is generated at block 510. Blocks 510-518 are repeatedly performed until there is a determination at decision point 516 that no further user input is needed.

At block 520, a determination as to whether a refill request is needed is made. The determination is based on the therapy, the regimen, and any received user input. If a refill request is needed, a refill request is generated at block 522. At block 524, fulfillment information corresponding to the refill request is received. In response to the receipt of fulfillment information, an event is generated at 510. Blocks 510-514, and subsequently, blocks 522 and 524 are performed periodically depending on the schedule and any received user input.

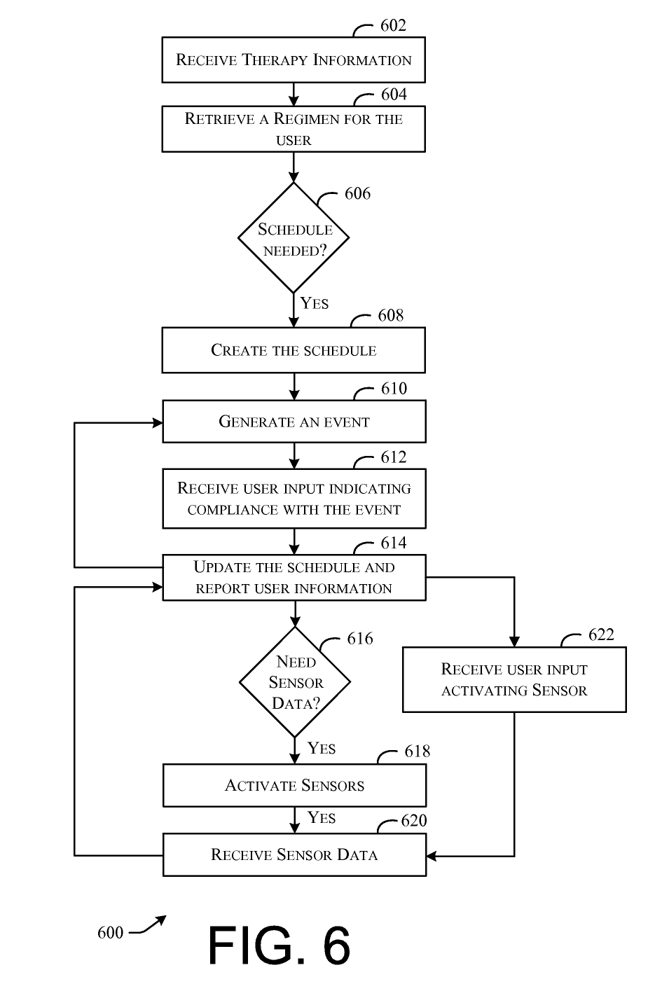

FIG. 6 depicts a block diagram 600 of still one further example method for using the therapy compliance engine 102. The method may begin at block 602 where therapy information is received. At block 604, a regimen is retrieved for the user. In at least one example, the retrieved regimen is based on the received therapy. Additionally, or alternatively, the regimen is based on participation by the user in a clinical trial. For instance, the regimen for a clinical trial may include more frequent vital sign readings than if the user was not participating in the clinical trial. In another example, the clinical trial regimen includes more frequently posing questions to the user in order to ascertain particular information. As a non-limiting example, a trial that is, at least in part, attempting to determine the amount of time required for a particular medication to take effect may pose frequent questions to the user regarding whether the user feels the effects at the time the question is posed. These questions may be posed more frequently in a clinical trial that they would be if the regimen has been based solely on a therapy.

At decision point 606, the therapy compliance engine makes a determination as to whether a schedule is necessary. This determination is based on information regarding the received therapy. If a schedule is needed, the method continues to block 608 where a schedule is created that specifies at least one situation for which at least one event associated with a therapy should be performed. At block 610, an event is generated. In at least one example, the event is determined by the regimen. At block 612, user input indicating compliance with the event is received. As discussed above, compliance is received from the user via a body-worn device or, alternatively, from a Bluetooth-enabled device. At block 614, the schedule is updated and information related to the user (e.g., information indicating user compliance) is reported. As described above, updating the schedule occurs because of received user input. At decision point 616, the therapy compliance engine determines whether sensor data is needed. This determination is based on the therapy and/or the schedule. If sensor data is determined to be necessary at block 616, one or more sensors are activated at block 618. Which sensors are activated depends on the therapy and/or the schedule. Sensor data is received at block 620. Based on the received sensor data, the schedule is updated at block 614. Blocks 614-620 are repeatedly performed until there is a determination at decision point 616 that no further sensor data is needed. Additionally, block 614-620 are performed periodically based on the schedule.

In at least one example, the user activates one or more sensors at block 622. The sensor data is received at block 620. Based on the received sensor data, the schedule is updated at block 614. The sequence of blocks 614, 622, and 620 may be performed any number of times. Additionally, the user may activate the sensors at various points in time. Sensor activation may occur at any suitable time and does not need to occur in the order illustrated in FIG. 6.

In at least one example, the schedule may be updated and the user information reported at block 614. Upon update of the schedule, an event may be generated at step 610. As discussed above, the event includes a reminder (e.g., a reminder to take medication), a request (e.g., a prescription request), and/or a question to be posed to the user (e.g., “Do you feel out of breath?”). The event is presented on the body-worn device either audibly, and/or textually. At block 612, user input indicating compliance with the event is received. At block 614, the schedule is updated and information related to the user (e.g., information indicating user compliance) is reported. The sequence of blocks 614, 610, and 612 may be performed any number of times. The sequence is performed, for example, due to the received user input indicating compliance and/or as a result of sensor data receipt.

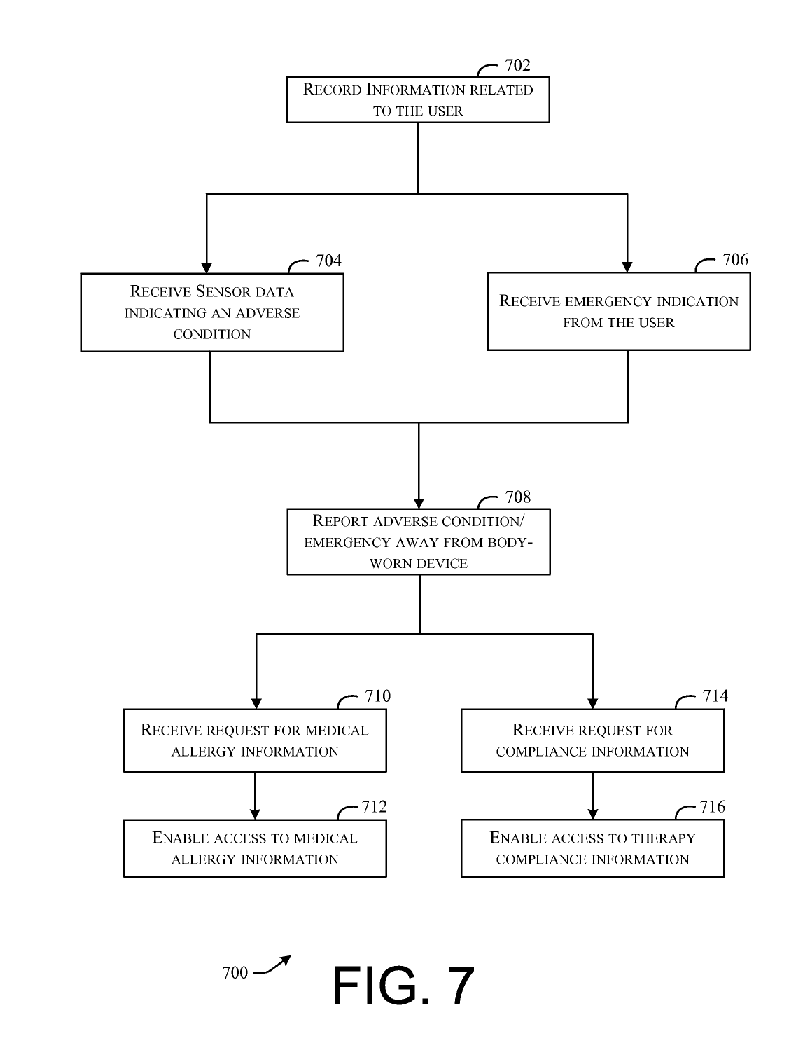

FIG. 7 depicts a block diagram of an example method 700 for using the therapy compliance engine in the context of an emergency situation. At 702, information is recorded that is related to the user. This information includes, but is not limited to, medical allergy information, physician contact information, emergency contact information, sensor data, and/or therapy compliance. The information may have been recorded in a manner described in FIGS. 5 and 6. At 704, sensor data is received that indicates an adverse condition to the user. For example, sensor data could indicate that the user's heart has stopped. Additionally, or alternatively, the device receives an indication of emergency from the user at 706. In one example, an emergency is initiated when the user presses a button, or combination or buttons, on the device. At 708, the adverse condition or emergency indication is reported away from the body-worn device. For example, the physician contact information and/or emergency contact information is utilized to notify the indicated contacts of the emergency. Similarly, the body-worn device reports the adverse condition and/or emergency to an emergency response unit. At 710, a request is received for medical allergy information. In one example, an emergency medical technician, or some other medical personnel attempts to access the medical allergy information on the body-worn device by using one or more user interfaces. The body-worn device, upon receiving the request, may enable access to the user's medical allergy information at 712. In at least one example, enabling access includes displaying the medical allergy information on the body-worn device. At 714, a request for therapy compliance information is received. In at least one example, a physician requests the therapy compliance information to ascertain what medication the user is prescribed and whether or not the user has been taken such medication. The body-worn device, upon receiving the request, may enable access to the user's medical allergy information at 716. In at least one example, enabling access includes displaying the therapy compliance information on the body-worn device. In another example, enabling access includes providing the information to a third-party website.

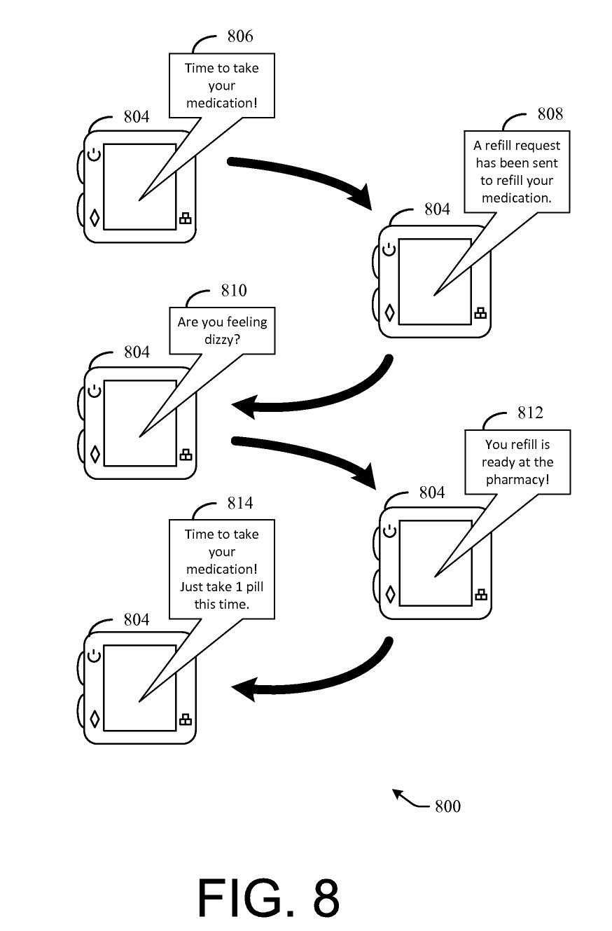

FIG. 8 depicts an example user interaction 800 in accordance with at least one embodiment. At 802, the user is presented, on a display screen 804 of the body-worn device 108, text 806. Based on input received from the user or, alternatively, from a Bluetooth device, the user is presented with text 808. The therapy implemented in the example interaction indicates that the user should be posed a question. The question comprises text 810, displayed on a display screen 804 of the body-worn device 108. Upon receipt of fulfillment information, a user is presented with text 812 via the display screen 804 of the body-worn device 108. Based on the example regimen, the user is presented with text 814 via the display screen 804 of the body-worn device 108.

Specific details are given in the above description to provide a thorough understanding of the embodiments. However, it is understood that the embodiments may be practiced without these specific details. For example, circuits may be shown in block diagrams in order not to obscure the embodiments in unnecessary detail. In other instances, well-known circuits, processes, algorithms, structures, and techniques may be shown without unnecessary detail in order to avoid obscuring the embodiments.

Implementation of the techniques, blocks, steps, and means described above may be done in various ways. For example, these techniques, blocks, steps and means may be implemented in hardware, software, or a combination thereof. For a hardware implementation, the processing units may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments may be described as a process which is depicted as a flowchart, a flow diagram, a swim diagram, a data flow diagram, a structure diagram, or a block diagram. Although a depiction may describe the operations as a sequential process, many of the operations can be performed in parallel or concurrently. In addition, the order of the operations may be re-arranged. A process is terminated when its operations are completed, but could have additional steps not included in the figure. A process may correspond to a method, a function, a procedure, a subroutine, a subprogram, etc. When a process corresponds to a function, its termination corresponds to a return of the function to the calling function or the main function.

Furthermore, embodiments may be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks may be stored in a machine-readable medium such as a storage medium. A code segment or machine-executable instruction may represent a procedure, a function, a subprogram, a program, a routine, a subroutine, a module, a software package, a script, a class, or any combination of instructions, data structures, and/or program statements. A code segment may be coupled to another code segment or a hardware circuit by passing and/or receiving information, data, arguments, parameters, and/or memory contents. Information, arguments, parameters, data, etc. may be passed, forwarded, or transmitted via any suitable means including memory sharing, message passing, token passing, network transmission, etc.

For a firmware and/or software implementation, the methodologies may be implemented with modules (e.g., procedures, functions, and so on) that perform the functions described herein. Any machine-readable medium tangibly embodying instructions may be used in implementing the methodologies described herein. For example, software codes may be stored in a memory. Memory may be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” may represent one or more memories for storing data, including read-only memory (ROM), random access memory (RAM), magnetic RAM, core memory, magnetic disk storage mediums, optical storage mediums, flash memory devices and/or other machine-readable mediums for storing information. The term “machine-readable medium” includes, but is not limited to, portable or fixed storage devices, optical storage devices, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

1. A method for managing therapy compliance with a body-worn device, comprising:
receiving a therapy for a user from a remote location, wherein the therapy specifies one or more treatments selected by a care provider;
retrieving a regimen for the user based on the therapy, the regimen specifying at least one situation for which to generate an event associated with the therapy;
generating a first event on the body-worn device, according to the regimen, wherein at least one of the therapy, the regimen, and/or the event is received by the body-worn device wirelessly, the body-worn device being worn on a user's limb;
receiving, by the body-worn device, user input affirmatively initiated by the user in response to the first event;
generating a second event on the body-worn device in response to the user input; and
wirelessly reporting information related to the user input away from the body-worn device by a cellular network.

2. The method for managing therapy compliance with the body-worn device of claim 1, further comprising:
generating a prescription request based on the recorded compliance;
detecting, by the body-worn device, an indication of fulfillment of the prescription request; and
notifying the user of the fulfillment.

3. The method for managing therapy compliance with a body-worn device of claim 1, further comprising:
determining a query to pose to the user based on the regimen;
posing the query to the user;
receiving additional user input, by the body-worn device, in response to the query; and
storing the additional user input.

4. The method for managing therapy compliance with a body-worn device of claim 1, wherein the user input indicating compliance with the event is received from a Bluetooth-enabled pill bottle.

5. The method for managing therapy compliance with a body-worn device of claim 1, further comprising:
causing a sensor on the body-worn device to collect vital sign information based on the regimen;
receiving the vital sign information; and
recording the vital sign information.

6. The method for managing therapy compliance with a body-worn device of claim 5, wherein the sensor on the body-worn device is at least one of a thermometer, a pedometer, a blood pressure monitor, a heart rate sensor, a blood-oxygen level sensor, a global positioning satellite sensor, or a glucose monitor.

7. The method for managing therapy compliance with a body-worn device of claim 5, further comprising:
modifying the regimen based on the received vital sign information; and
generating an additional event on the body-worn device, specified by the modified regimen.

8. A body-worn device for managing therapy compliance, comprising:
one or more processors; and
one or memories coupled with said one or more processors, wherein the one or more processors and one or more memories are configured to:
receive a therapy for a user from a remote location, wherein the therapy specifies one or more treatments selected by a care provider;
retrieve a regimen for the user based on the therapy, the regimen specifying at least one situation for which to generate an event associated with the therapy;
generate a first event on the body-worn device, according to the regimen, wherein at least one of the therapy, the regimen, and/or the event is received by the body-worn device wirelessly, the body-worn device being worn on a user's limb;
receive, by the body-worn device, user input affirmatively initiated by the user in response to the first event;
generate a second event on the body-worn device in response to the user input; and
wirelessly report information related to the user input away from the body-worn device by a cellular network.

9. The body-worn device for managing therapy compliance of claim 8, wherein the one or more processors and one or more memories are further configured to:
receive, from a sensor on the body-worn device, vital sign information indicating an adverse health condition related to the user; and
wirelessly report the vital sign information away from the body-worn device.

10. The body-worn device for managing therapy compliance of claim 8, receive, by the body-worn device, an indication of emergency status, wherein the indication of emergency status is initiated by the user.

11. The body-worn device for managing therapy compliance of claim 8, wherein the one or more processors and one or more memories are further configured to:
cause a sensor on the body-worn device to collect vital sign information based on the regimen;
receive the vital sign information; and
record the vital sign information.

12. The body-worn device for managing therapy compliance of claim 11, wherein the one or more processors and one or more memories are further configured to:
cause the sensor on the body-worn device to collect additional vital sign information from the sensor on the body-worn device;
receive the additional vital sign information; and
record the additional vital sign information.

13. The body-worn device for managing therapy compliance of claim 8, wherein the one or more processors and one or more memories are further configured to:
receive medical allergy information related to the user; and
display the medical allergy information related to the user on the body-worn device.

14. The body-worn device for managing therapy compliance of claim 8, wherein the one or more processors and one or more memories are further configured to:
receive a request for information related to the user; and
in response to the request, enable access to the information related to the user.

15. A non-transitory computer-readable storage medium for managing therapy compliance with a body-worn device having stored thereon computer-executable instructions that, when executed by a processor, cause the processor to perform operations comprising:
receiving a therapy for a user from a remote location, wherein the therapy specifies one or more treatments selected by a care provider;
retrieving a regimen for the user based on the therapy, the regimen specifying at least one situation for which to generate an event associated with the therapy;
generating a first event on the body-worn device, according to the regimen, wherein at least one of the therapy, the regimen, and/or the event is received by the body-worn device wirelessly, the body worn device being worn on a user's limb;
receiving, by the body-worn device, user input affirmatively initiated by the user in response to the first event;
generating a second event on the body-worn device in response to the user input; and
wirelessly reporting information related to the user input away from the body-worn device by a cellular network.

16. The non-transitory computer-readable storage medium for managing therapy compliance with a body-worn device of claim 15, wherein the regimen is further based on participation by the user in a clinical trial.

17. The non-transitory computer-readable storage medium for managing therapy compliance with a body-worn device of claim 15, further comprising:
receiving vital sign information from a sensor on the body-worn device; and
recording the vital sign information.

18. The non-transitory computer-readable storage medium for managing therapy compliance with a body-worn device of claim 17, further comprising:
receiving additional vital sign information from the sensor on the body-worn device; and
recording the additional vital sign information.

19. The non-transitory computer-readable storage medium for managing therapy compliance with a body-worn device of claim 18, further comprising:
wirelessly reporting the vital sign information and the additional vital sign information away from the body-worn device;
modifying the regimen based on the received vital sign information or received additional vital sign information; and
generating an additional event on the body-worn device, specified by the modified regimen.

20. (canceled)

21. The method for managing therapy compliance with the body-worn device of claim 1, further comprising:
modifying the regimen to include the second event in response to the user input affirmatively initiated by the user, wherein generating the second event on the body-worn device is based on the modified regimen.
